(19) United States
(12) Patent Application Publication
(10) Pub. No.: US 2018/0092521 A1
(43) Pub. Date: Apr. 5, 2018

(54) DEVICE, SYSTEM AND METHOD FOR ILLUMINATING A STRUCTURE OF INTEREST INSIDE A HUMAN OR ANIMAL BODY

(71) Applicant: KONINKLIJKE PHILIPS N.V., EINDHOVEN (NL)

(72) Inventors: JULIAN CHARLES NOLAN, PULLY (CH); MATTHEW JOHN LAWRENSON, Bussigny-pres-de-lausanne (CH); JUERGEN WEESE, Norderstedt (DE)

(21) Appl. No.: 15/561,351

(22) PCT Filed: Mar. 24, 2016
(86) PCT No.: PCT/EP2016/056475

§ 371 Date: Sep. 25, 2017

(30) Foreign Application Priority Data
Mar. 26, 2015 (EP) 15161043.3

(51) Int. Cl. A61B 1/06; A61B 5/00; G02B 23/24; A61B 1/00

(52) U.S. Cl. CPC A61B 1/0638; A61B 1/00009; G02B 23/2461; A61B 5/0084

(57) ABSTRACT

A medical system for illuminating a structure of interest inside a human or animal body, the medical system comprising: i) a medical device comprising a controllable light source according to any of the preceding claims, characterized in that said medical system comprises: ii) a control unit for generating a control signal for controlling the controllable light source, wherein the control unit comprises, iii) a receiving unit configured to receive data of the human or animal body, iv) a further receiving unit (266, 366) configured to receive stored information from a memory (261, 361), said memory configured for storing information as to i) a depth of a structure of interest (220, 320) within a tissue and/or a cavity of the human or animal body, and ii) the wavelength, or range of wavelengths suitable for illuminating the structure of interest (220, 320) based on the depth of said structure of interest (220, 320) within the tissue and/or the cavity, whereby the control unit is arranged to generate, based on the received data and stored information, a control signal capable of being received by the controllable light source so that said controllable light source illuminates the structure of interest at a wavelength, or a range of wavelengths suitable to illuminate the structure of interest.

### FIELD OF THE INVENTION

The invention relates to a medical system for illuminating a structure of interest inside a human or animal body, the medical system comprising a medical device comprising i) a controllable light source configured to emit light with a selected wavelength, or a selected range of wavelengths, selected from a range of wavelengths, and ii) an input for receiving a control signal emitted by a control unit, wherein the medical system further comprises a control unit for generating the control signal for controlling the controllable light source.

The invention also relates to a method for illuminating a structure of interest inside a human or animal body.

The invention also relates to a computer program comprising program code means for causing, when executed, a medical system for illuminating a structure of interest inside a human or animal body, to carry out the steps of the method according to the present invention.

### BACKGROUND OF THE INVENTION

Minimally invasive surgery is often performed using elongated instruments inserted into a patient's body through small ports. Endoscopy usually refers to the action of looking inside the human or animal body through such small ports by means of an endoscope. For a surgery involving an endoscope, an endoscopic camera is generally inserted into a port so to provide visualization of the surgical site, or any other sites inside the human or animal body.

An endoscope is an instrument used to examine the interior of a hollow organ cavity, which may be used for surgery. Many types of endoscopy are known depending of the region of the body that is under examination. To this extent, specialized endoscopes are named for where they are intended to look, for examples a cystoscope (bladder), a nephroscope (kidney), a bronchoscope (bronchi), a laryngoscope (larynx), an otoscope (ear), an arthroscope (joint), a laparoscope (abdomen), and a gastrointestinal endoscope. Generally, endoscopes comprise a rigid or flexible tube, a light delivery system (or light source) to illuminate the object under inspection, a lens and a camera (or eyepiece).

The light source is normally outside the body as the light is typically directed via an optical fiber system, where a lens system for transmitting the image from the objective lens to the viewer is generally proposed. Typically endoscopes comprise a relay lens system in the case of rigid endoscopes or a bundle of optical fibers in the case of a fiberscope, following which a camera transmits image to a screen for image capture. Endoscopes generally contain an additional channel to allow entry of medical instruments or manipulators.

The light source of the endoscope is configured to illuminate a region inside the body, where the emitted light reflects on the tissue such that an image is captured by the camera, and then displayed on a display. Endoscopes are generally controlled by the physician based on the image represented on the display.

By the use of camera, recent endoscopes allow for enhanced real time viewing of the surgical site, and allow for digital image capture for later analysis by a physician or surgical team. As the sophistication and quality of the digital camera systems have been increased, the ability to image internal features of the human body with greater precision and accuracy is improved. This precision and accuracy is required for observation of highly compact and complex surgical sites. Moreover, the use of camera system for receiving given emitted wavelength enables detection of structure(s) occluded to the naked eye, for example behind the wall of a cavity.

A medical bio-imaging apparatus for allowing the viewing of and direction of laser energy toward structures located behind occluding materials such as haze, smoke, tissues and/or blood is known from US 2006/0106282. The latter discloses a technology to produce wavelengths of light within the infrared spectrum in order to view surgical sites normally occluded by conditions such as smoke, fluids, tissues, and/or haze and to direct laser energy to the viewed surgical site. The invention also allows selected wavelengths of infrared light to be directed for the purpose of illumination and imaging of a surgical site. The invention additionally allows laser energy to be accurately directed to the imaged site. In use, light can enter a body of an endoscope or similar device through a light channel such that a specific wavelength can be selected that is different from conventional illumination wavelengths. The selection of a specific range of infrared wavelengths can be accomplished by use of filters and/or gratings located on a light source such as a flashtube or on such devices as a filter wheel. Alternatively, a continuously variable filter wheel may be used in order to select the desired wavelength of light.

It is a drawback of known endoscopes that the wavelengths emitted by the light source are not tailored to the region to observe such that the best possible illumination of the region to observed is provided to the physician so as to generate a further increased precision and accuracy of the image. Thus, the efficacy of the endoscopy is prone to mistakes.

### SUMMARY OF THE INVENTION

It is an object of the invention to provide a medical system of the kind set forth in the opening paragraph which enables a better illumination of a structure of interest such that a more accurate assessment (or diagnostic) of a structure is provided.

The invention makes use of a medical device as defined in the opening paragraph wherein the controllable light source is further configured to illuminate the structure of interest at a wavelength, or a range of wavelengths suitable to illuminate the structure of interest, wherein said wavelength, or a range of wavelengths being chosen from the selected wavelength, or the selected range of wavelengths, based on the received control signal.

According to the invention, the foregoing object is realized by a medical system defined in the opening paragraph characterized in that a control unit comprising a) a receiving unit configured to receive data (for instance image data) of the human or animal body, a further receiving unit configured to receive stored information from a memory, said memory configured for storing information as to i) a depth of a structure of interest within a tissue and/or a cavity of the human or animal body, and ii) the wavelength, or range of wavelengths suitable for illuminating the structure of interest based on the depth of said structure of interest within the tissue and/or cavity, whereby the control unit is arranged to generate, in based on the received data and stored information, a control signal capable of being received by the controllable light source such that said controllable light source is arranged to emit light at a wavelength, or a range of wavelengths suitable to illuminate the structure of interest based on said control signal.

The medical system according to the present invention is advantageous in that the control signal to be outputted by the control unit enables selection of the wavelength, or range of wavelengths (for instance by the system, or by the controllable light source) as described above such that the structure of interest is illuminated so as to be made visible (or detectable) to a user. Such illumination of the structure of interest, according to the present invention, is possible even when said structure is occulted by the tissue (for instance an ensemble of cells, for instance a cavity wall), by fluids, or any other element. In other words, the control signal enables selection of a wavelength or a range of wavelengths from a plurality of wavelengths capable of being emitted by the controllable light source such that the object of the invention is realized.

The medical system according to the present invention is further advantageous as it enables the use of robust information correlated to each other as describe above, such that the control signal is generated based on such robust information. The memory enables the storage of information, in the form of data for example, which relates to the structure of interest). Such information can have been previously uploaded in the memory such that the system may retrieve the needed information, which may be indicative of the depth of the structure relative to the wall of a cavity for example (or any wall of a human or animal tissue) as well as to the wavelength or range of wavelengths optimal (or advisable, or adequate) to illuminate such structure in given tissue at a given dept. Therefore, by the correlation of the information stored in the memory, a wavelength or range of wavelengths to be chosen for illuminating the structure of interest is selected (or chosen, or determined). Such information is embedded into the control signal and convey by means of said control to the relevant features (for instance the controllable light source) signal such that light at the desired wavelength or range of wavelengths is emitted.

The medical system according to the present invention is further advantageous as it enables better visual assessment of a tumor when a marker (for instance a contrast agent, for instance a fluorescent marker) is inserted into said tumor such that emphasis on the contour (or the rim) of said tumor is provided. In this situation, a medical system according to the present invention enables to get an optimal response from such marker as the wavelength, or the range of wavelengths to be emitted is optimal to get the response from such marker (e.g.: in the case of a fluorescence marker, enables to excite the electron at the most optimal wavelength) such that a user can adequately see, and make an analysis thereon.

The invention further enables accurate observation of a structure or structures (for instance a cyst, a tumor, or a blood vessels) which are usually occulted by a tissue, an organ, or a cavity. A control signal enables the controllable light source to emit light at an appropriate wavelength, or range of wavelengths such that a generally occulted structure can be detected swiftly and automatically. The control signal is configured to receive all necessary information as to automatically select the appropriate wavelength, or range of wavelengths based on data as it will be further elucidated hereinafter. The medical device can therefore directly select the appropriate wavelength, or range of wavelengths, for illumination of the structure to be observed, thereby enabling a user (for instance a skilled user, for instance a physician, for instance a surgeon) to see (for instance on a display) said structure of interest (corresponding to the region of interest on an image data). In other words, by providing a control signal to the medical device according to the present invention, several steps that require manual labor can be automated, such that the appropriate wavelength, or range of wavelengths illuminate the structure of interest.

The invention is further advantageous in that detection of generally occulted structure(s) or region(s) is of critical importance for surgery, or diagnostic purposes is made possible. Minimally invasive surgeries should be quick, precise and absent of any complication. To this extent, it is desired to have reliable means to see the procedure, therefore limiting as much as possible the reliance on external sources (such as a previously generated image from any modality). In order to see the procedure, as referred previously, it is needed to adequately illuminate the area, and more precisely the structure of interest, such that, for example, adequate removal of said structure occurs, or an appropriate sample of said structure is extracted.

The invention is further advantageous in that it enables automatic adjustment of the controllable light source such that an optimal, or a finest, or a suitable, or an adequate wavelength, or range of wavelengths are used for illumination of the structure of interest, thereby minimizing errors, and optimizing the image quality (including the contrast) of any image to be displayed by a medical system according to the present invention.

In an embodiment, the medical system comprises a medical device comprising a detection unit configured to receive reflected emitted light from the illuminated structure of interest, and to generate a detected signal. This arrangement is advantageous in that it enables numeration (e.g. digitally) of the reflection of the illuminated structure of interest such that a detected signal is generated and outputted to the system. Said signal can be processed by appropriate means and/or algorithm(s) such that a medical image of the illuminated structure of interest is displayed on a display, enabling a user to see said structure of interest. This arrangement is further advantageous in that it enables miniaturization of the medical devices, obviating need for means (such as mirrors) for enabling the user (for instance a skilled user, for instance a physician, for instance a surgeon) to directly see within the device.

In another embodiment, the controllable light source of the medical device according to the present invention comprises a light-emitting diode (LED), or a distal end of an optical fiber (or a group of optical fibers). This arrangement is advantageous in that it enables for a reliable, easy to control and robust controllable light source, which can fulfill the criteria of the present invention. This arrangement is further advantageous in that a range of wavelengths (the plurality of wavelengths) can be emitted throughout a wide spectrum of wavelength, thereby enabling a suitable wavelength or suitable range of wavelengths is chosen from this plurality of wavelengths. The latter permitting illumination of different types of tissues such that the structure of interest is detectable (visible).

According to the present invention, a tissue comprises a biological tissue, therefore, for instance an ensemble of cells that carry a function. An organ comprises a collection of tissues joined in a structural unit to serve a common function. A cavity comprises any fluid-filled space in a multicellular organism.

In an embodiment, the received data by the medical system according to the present invention comprise region information such that the region of interest is determined, for example by being previously processed by an image segmentation algorithm. This arrangement is advantageous in that the received data do not have to be further processed by the system, for example, the depth of the region of interest relative to the wall of an imaged organ is identified such that the structure of interest is inferred, thereby improving speed of the medical system according to the present invention.

In an embodiment, the medical system according to the present invention further comprises a correlation unit configured to correlate the received data from the receiving unit and the information stored in the memory such that the depth and wavelength, or range of wavelengths are correlated, thereby generating correlation data, wherein the control unit is arranged to generate, based on the correlated information, the control signal. This arrangement is advantageous in that it enables the system to automatically assess the depth of the structure of interest relative to the surface of the tissue capable of being illuminated (or relative to any other fix element, for instance the head of the medical device according to the present invention). Consequently, the determination, hence the calculation, can be achieved within the system such that the control signal embeds and conveys all necessary information to control (to regulate) the controllable light source. In other words, the system according to this embodiment enables a smaller and less complex medical device to be coupled by the system, as all the processing of information is achieved in the medical system, alleviating, at least partially, the need of a control unit in the medical device.

In an embodiment, the memory of the medical system according to the present invention comprises a first look-up table comprising information as to the depth of the region of interest relative to a surface of a tissue capable of being illuminated by the controllable light source, and a second look-up table comprising information as to the wavelength, or range of wavelengths that is most suitable for illuminating the structure of interest based on the depth of the determined region of interest relative to the tissue capable of being illuminated by the controllable light source. This arrangement is advantageous in that it provides a robust means to convey information to be correlated such that a desired output (i.e.: control signal) is generated. By the use of two distinct look-up tables, the system can be updated efficiently with new information such that the information therein and used is up to date. This arrangement is further advantageous in that it provide the possibility to link different look-up tables which are physically stored at different locations, thereby suiting the specific needs of a given health center.

In an embodiment, the received data by the medical system according to the present invention consists of image data of the region of interest that have been processed by one or more mathematical models prior to be received by the receiving unit, such mathematical model arranged for recognizing and/or segmenting the region of interest in image data such that a region of interest is identified. This arrangement is advantageous in that it enables proper detection and/or identification of a region of interest in image data such that the system can use robust information as to assess, for example, the depth of such region of interest inside the tissue, or the cavity. Such model(s) enables proper detection of a region of interest of image data thereby increasing reliability of the system according to the present invention.

In an embodiment, the received image data of the medical system according to the present invention have been processed by one or more deformable models such that a contour of the region of interest within an organ is identified. The use of deformable models in image data has been detailed in Weese J., Wachter-Stehle I., Zagorchev L., Peters J., Shape-Constrained Deformable Models and Applications in Medical Imaging, Lecture Notes in Computational Vision and Biomechanics Volume 14, 2014, pp 151-184. Such deformable models, for instance a segmentation algorithm, for instance statistical shape model for 3D image enables a flexibility of active contour approaches such that the depth of the structure of interest relative to a wall capable of being illuminated by the controllable light source is easily identifiable. By the use of these deformable models, a determination of a depth distance (relative to a wall capable of being illuminated by the light emitted by the controllable light source) can be determined, or inferred, or calculated as it will be further elucidated hereunder.

In an embodiment, the medical system according to the present invention further comprises a display for displaying a visualization of the detected signal generated by the medical device. This arrangement is advantageous in that it enables any user and/or patient and/or caregiver to see the structure of interest without the need of directly looking within the medical device. This arrangement is further advantageous in that it provides for improved image quality, thereby enabling adequate assessment and/or diagnostic by the user (for instance a skilled user, for instance a physician, for instance a surgeon).

In an embodiment, the medical system is further arranged to receive a location signal indicative of the location of the controllable light source, the medical system further comprising an alarm signal for emitting an alarm when the received location signal indicates that the controllable light source is positioned to illuminate the structure of interest. This arrangement is advantageous in that as a consequence of the structure of interest is occulted (or hidden) by a tissue, or a wall, or a cavity, the wavelength and/or range of wavelengths enabling visual detection of said structure of interest may not be adequate for displacement (or movement) of the medical device into the human or animal body. This embodiment proposes the generation of an alarm signal when the controllable light source is the suitable location to illuminate the (occulted) structure of interest, thereby enabling guiding of said medical device within the human or animal body with a range of wavelengths suitable for such act. The user (or the robot) will be triggered to stop (or to limit) any movement following the emission of the alarm signal such that the controllable light is to be located to illuminate the structure interest. Such alarm may be for example a visual signal, an audio signal, or any other means so as to grasp attention of the operator (or the user), or to provide for an input to a robot such that action(s) is taken.

In an embodiment, the database of the system according to the present invention is further arranged to host a mathematical model configured to run a correlation analysis on the information contained in said memory such that the control unit emits a control signal indicative of the correlated analysis. From this analysis, the information as to i) the depth of the region of interest, 2) the type of tissues (or organ) in which the region of interest is located, and 3) a wavelength or range of wavelengths suitable to penetrate the tissue in which the region of interest is located such that the region of interest is detectable. This embodiment is advantageous in that generation of the control signal follows correlation of information such as the tissues, the depth of the structure of interest into a given tissue and the optical property of said tissue, as previously tested, thereby enabling the controllable light source to emit light at a wavelength or range of wavelength that is optimal based on numerous factual analysis from the image data for instance.

In another embodiment, the medical system according to the present invention comprises a location unit configured to locate the medical device from received reflected emitted light from an illuminated tissue of the human or animal body, such that a location of said tissue is locatable based on the received reflected emitted light. This arrangement is advantageous in that it enables (partial) robotisation such that a medical device according to the present invention can be remotely controlled by a skilled user. This arrangement further is advantageous in that is enables automatic detection of the location (i.e.: a particular place or position) of the medical device inside the cavity. In other words, by this arrangement, a system can detect the location of the controllable light source such that it can be precisely assess with, for example, coordinate, such that proper movement of the medical device can be made (automatically, or manually) such that the controllable light source reaches a position where illumination of the structure of interest is possible. For instance, a light sensor detects light meaning from the target tissue to provide a detected signal, which can be further analyzed such that the location of the light source inside the human or animal body is identified.

In another embodiment, the location unit of the medical system according to the present invention comprises a spectrometer for obtaining a measured data from reflected emitted light from an illuminated tissue of the human or animal body, such measured data representative of an optical spectrum of the illuminated tissue of the human or animal body; wherein the location unit is further configured to emit a location signal indicative of the location of the tissue of the human or animal body based on the measured data, said location signal capable of being interpreted by the control unit such that the location of the tissue is determined based on the optical spectrum of the illuminated tissue. This arrangement is advantageous in that it enables a robust means to automatically detect the location of the controllable light source.

According to a third aspect of the invention, the foregoing object is realized by a method characterized in the steps of a) receiving image data of the human or animal body where a region of interest is identified; b) determining a suitable wavelength or range of wavelengths based on correlated information contained in a memory as to i) a depth of a structure of interest within a tissue and/or a cavity of the human or animal body, and ii) the wavelength, or range of wavelengths suitable for illuminating the structure of interest based on the depth of said structure of interest within the tissue and/or the cavity; c) outputting an control signal based on the correlated information stored in the memory, the control signal capable of being received by the controllable light; d) controlling the controllable light source based on the control signal to emit light at a wavelength, or range of wavelengths suitable to illuminate the structure of interest, said wavelength, or a range of wavelengths being chosen from the plurality of wavelengths.

The above-described method provides similar, or the same benefits as the medical system according to the second aspect of the invention. This method, when used in a system comprising the different embodiments described above, has similar advantages as the corresponding embodiments of the system. The proposed method is advantageous in that it enables a medical system according to the invention so as to enable illumination of a structure of interest by a controllable light source with the wavelength, or the range of wavelengths suitable for a user to see (via the means of a display for instance) the structure of interest.

According to a fourth aspect of the invention, at least one of the aforesaid objects is realized by a computer program comprising program code means for causing, when executed, a medical system for illuminating a structure of interest inside a human or animal body to carry out the steps of the method as defined above. This arrangement is advantageous as it enables automation of the method discussed above on a medical system according to the present invention. For example, the computer program permits a robot, or any other machines and/or devices to proceed with the steps of the method described above. Automation is advantageous in that it enables quicker surgery, and alleviate the risk of human mistakes. Moreover, it enables surgery from a remote location, which is beneficial for patients as skilled doctors (or physicians) may proceed with a (minimally invasive) surgery by the system according to the present invention regardless of their geographical situation.

These and other aspects of the invention are apparent from and will be elucidated with reference to the embodiments described hereinafter.

It will be appreciated by those skilled in the art that two or more of the above-mentioned options, implementations, and/or aspects of the invention may be combined in any way deemed useful.

### BRIEF DESCRIPTION OF THE DRAWINGS

These and other aspects of the applicator device, the system and the method according to the invention will be further elucidated and described with reference to the drawing, in which:

FIG. 1 schematically shows an example of a medical device for use in a medical system according to the present invention.

FIG. 2 schematically shows an embodiment of a system according to the present invention.

FIG. 3 schematically shows a further embodiment of a system according to the present invention.

FIG. 4 schematically shows an embodiment of a method according to the present invention.

### DETAILED DESCRIPTION OF EMBODIMENTS

Certain embodiments will now be described in greater detail with reference to the accompanying drawings. In the following description, like drawing reference numerals are used for like elements, even in different drawings. The matters defined in the description, such as detailed construction and elements, are provided to assist in a comprehensive understanding of the exemplary embodiments. Also, well known functions or constructions are not described in detail since they would obscure the embodiments with unnecessary detail. Moreover, expressions such as “at least one of”, when preceding a list of elements, modify the entire list of elements and do not modify the individual elements of the list.

A medical device according to the present invention, for instance an endoscope, is configured to be inserted into the human or animal body. Such medical device comprises a light source for illumining, or for emitting light inside the human or animal body, thereby allowing examination of the interior of the human or animal body, for instance an organ (preferably a hollow organ), or a cavity. The medical device according to the present invention is configured to enable examination of a structure of interest (for instance a tumor, for instance blood vessels) which would generally be occulted by a wall (for example the outside wall of a cavity, when outside refers to the side capable of being illuminated by the light source of known endoscopes).

Illumination by a light (electromagnetic radiation) of a particular wavelength, or range of wavelengths can be beneficial for imaging through turbid or opaque materials since different materials will typically have different light scattering and reflective properties depending on the wavelength(s) absorbed. Tissues, fluids cause visual distortion and opacity due to the light scattering properties of these elements. These differing absorptive and reflective properties of fluids and/or tissues often mean that such materials be made essentially transparent (for instance translucent) at a certain selected wavelength or range of wavelengths, which are often unique to that fluid or tissue. Based on such properties, tissues can be identified based on the differing optical properties they possess when illuminated at differing wavelengths of light.

For example, a recent study confirmed that specific tissues of the human body (for example heart tissue, liver tissue, kidney tissue) have different properties (for instance depth penetration of the wavelength(s) of the emitted light) for different wavelengths (nm). The following table represents experimental data of measurements of optical properties of in vivo (μa and μs′) human tissue at room temperature (22° C) (Sandell J. L., Zhu T. C., A review of in-vivo optical properties of human tissues and its impact on PDT, J Biophotonics. 2011November; 4(11-12): 773-787):

In vivo optical properties at commonly used treatment wavelengths for PDT

| | | | | Experimental | |
|---|---|---|---|---|---|
| Tissue | λ (nm) | μₐ (cm⁻¹) | μₛ′ (cm⁻¹) | Method | References |
| Bladder | 532 | 0.27-0.71 | 1.28-3.30 | 4 | [108] |
| | 630 | 0.28-0.76 | 2.5-6.37 | 4 | [108] |
| Bone | 650 | 0.09-0.14 | 12.5-15.8 | 1 | [109] |
| | 760 | 0.07-0.09 | 11.9-14.1 | 1 | [109] |
| Brain | 420 | 0.01-3.51 | 8.75-55.83 | 4 | [108] |
| | 532 | 0.02-3.84 | 0.10-46.3 | 4 | [108] |
| | 630 | 0.02-0.50 | 3.72-21.97 | 4 | [108, 110, 111] |
| | 760 | 0.11-0.17 | 4.0-10.5 | 1, 2 | [6, 112] |
| | 780 | 0.078-0.089 | 8.42-9.16 | 2 | [8] |
| Breast | 660 | 0.037-0.110 | 11.4-13.5 | 1, 4 | [113-118] |
| | 760 | 0.031-0.10 | 8.3-12.0 | 1, 4 | [115, 116, 118-121] |
| | 900 | 0.096-0.29 | 3.33-5.86 | 1, 4 | [9, 115, 118, 122] |
| Tumor | 530 | 0.60-0.86 | 28.0-32.1 | 4 | [123] |
| | 690 | 0.070-0.10 | 14.7-17.3 | 2 | [124] |
| | 895 | 0.068-0.102 | 12.4-13.1 | 2 | [124] |
| Bowel | | | | | |
| Small | 630 | 0.19-0.21 | 8.95-10.05 | 4 | [35, 76] |
| Large | 630 | 0.12-0.18 | 10.11-10.42 | 4 | [35, 76] |
| Diaphragm | 661 | 0.15-1.08 | 9.65-21.7 | 4 | [125] |
| Heart | 630 | 0.03-1.55 | 17.56-75.06 | 4 | [126] |
| | 661 | 0.12-0.18 | 5.22-90.80 | 4 | [125] |
| Liver | 630 | 1.15-1.56 | 21.6-30.4 | 4 | [35, 76] |
| Lung | 630 | 0.16-1.36 | 1.07-83.81 | | [126] |
| | 661 | 0.49-0.88 | 21.14-22.52 | 4 | [125] |

As further elucidated hereunder, based on the above, the skilled person will see that following pre-identification of a region of interest from data (for instance image data) generated by an imaging system modality (for instance X-Ray, CT, MR, Ultrasound), a medical device according to the present invention may receive control data (for example a specific wavelength, for example a range of wavelengths) determined namely by such pre-identified region of interest information, such that a controllable light source (for instance a LED, for instance a plurality of laser, for instance the tip of an optical fiber, for instance the respective tip of a plurality of optical fibers) emits a light signal dependent on such received control data.

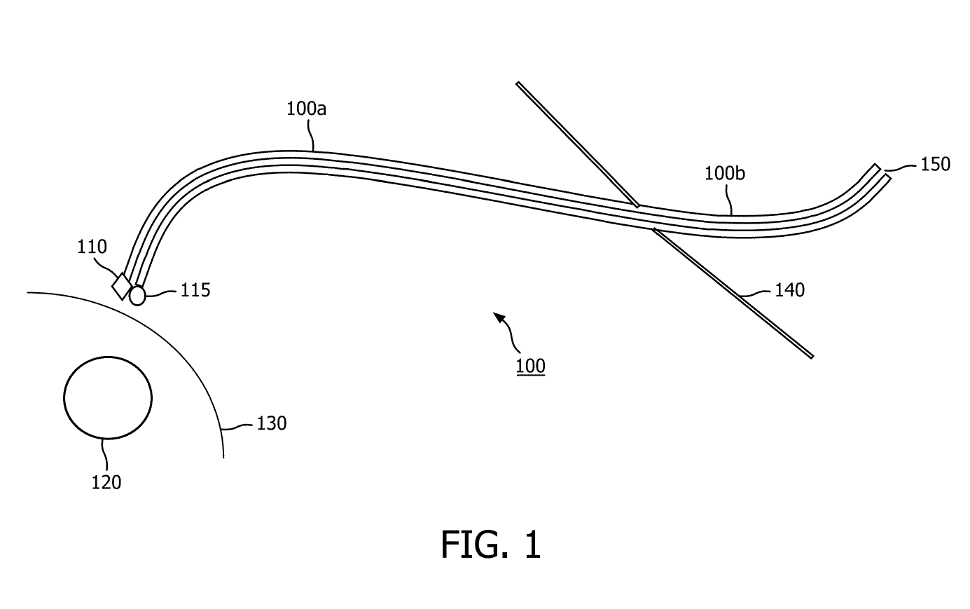

FIG. 1 schematically shows an example of a medical device 100 (for instance an endoscope) for use in a medical system according to the present invention. Said medical device comprises a proximal part 100a configured to be inserted inside the human or animal body and a distal part 100b configured to remain outside the human or animal body. The medical device 100 is inserted into the human or animal body via a cut on the skin 140 of the human or animal undergoing a surgery. Said distal part 100b is configured to enable connectivity with a system, for instance a system according to the present invention, for instance a camera system, for instance a robotic system, for instance a computer system, or alternatively enabling maneuver of the medical device 100 by a user (for instance a skilled user, for instance a physician, for instance a surgeon). The distal part 100b of the medical device 100 comprises one or more input(s)/output(s) 150 such that the medical device 100 can be in communication (for instance connected, or alternatively by means of wire(s), or alternatively wirelessly) with the system according to the present invention, or alternatively a camera system, or a robotic system, or a computer system, or any other means suitable to provide or receive information from the medical device 100.

The medical device 100 according to the present invention comprises a controllable light source 110, and may additionally comprise a sensor 115 on the proximal part of the medical device 100a. The controllable light source is a light source arranged to emit light at a plurality of wavelengths, for instance in the visible spectrum, additionally or alternatively in the ultraviolet spectrum, additionally or alternatively in the infrared spectrum. By controllable light source 110, one should read that a selected wavelength or range of wavelengths is selectable from the plurality of wavelengths that can be emitted by the controllable light source 110. Reactive of a control signal, the controllable light source 110 is configured to emit light at a chosen and/or at a specific wavelength, or at a chosen and/or at a specific range of wavelengths, such wavelength or range of wavelengths being selectable within the total range of the plurality of wavelengths spectrum capable of being emitted by the controllable light source 110.

In an exemplary embodiment, the controllable light source 110 consists of a LED arranged to emit a light signal at any wavelength between 400 and 730 nm. Following reception of a control signal instructing the emission of a wavelength of 530 nm, the controllable light source according to this exemplary embodiment will emit light at the desired wavelength of 530 nm.

In an alternative embodiment, the controllable light source 110 consists of one or more optical fibers for transmitting a light signal at any wavelength suitable for the purpose of the present invention. The origin of the light signal thereby transmitted may therefore be outside the body, for instance within the medical system (not shown) or coupled to the medical system (not shown) such that the light signal is guided by one or more optical fibers within the medical device 100 to the controllable light source 110.

The medical device 100 according to the present invention is configured to emit light towards a surface 130 such that said surface becomes illuminated by such emitted light. This surface 130 can be for example a wall of a cavity, alternatively a wall of a hollow organ; in other words, said surface 130 can be any surface of the human or animal body capable of being illuminated by a light source, for instance the controllable light source according to the present invention.

The surface 130, for example a human tissue, for instance the tissue around an organ, is substantively opaque, or alternatively translucent such that any structure of interest 120 (for instance a tumor, for instance a blood vessel) situated behind this surface 130 relative to the controllable light source 110 is blocked from receiving a plurality of wavelengths from the light emitted by the controllable light source 110 when said light source emits light at a plurality of wavelengths simultaneously, for instance a white light. Consequently, said structure of interest 120 is not visible to the user when illuminated by a plurality of non-specific wavelengths, as the vast majority of the emitted light is reflected by the surface 130.

As further elucidated below, such structure of interest may be visible to the user when illuminated by a specific wavelength, or range of wavelengths, such as made possible with a medical device according to the present invention. The medical device is configured for receiving a control signal from a processor (not shown in FIG. 1) via the one or more input(s)/output(s) 150. Said control signal, as explained above enables the controllable light source 110 to emit light at an appropriate, or suitable wavelength or range of wavelengths such that the structure of interest 120 becomes visible notwithstanding the surface 130.

Additionally, or alternatively, a sensor 115, for instance a detection unit, is arranged to receive reflected light from the structure of interest 120 such that a detection signal is generated and outputted from the medical device 100 via the one or more input(s)/output(s) 150. Said sensor 115 can be for instance an image detector, comprising for instance a CCD, DMOS, APS or any other imaging chip known in the art that would, without modification, be suitable to be used as a sensor 115 in the medical device 100 according to the present invention.

Additionally, or alternatively, the sensor 115 further comprises a detection unit configured to receive reflected light from the wall 130 and/or the structure of interest 120 and/or other structure occulted by the wall as originally emitted by the controllable light source 110. Such reflected light enables detection of the wall 130, structures interest 120 thereby illuminated. The detection unit is configured to generate a detection signal, which can be processed by the system according to the present invention as further described hereunder. Said detection signal convey information relative to the detecting, or the finding, or the assessing, or the inferring of the location of the controllable light source 110 inside the human or animal body. The skilled person will understand that once the controllable light source is located at the proper location inside the human or animal body, the wavelength or range of wavelengths based on the control signal can be emitted by the controllable light source such that the emitted wavelength or range of wavelengths can reach the structure of interest 120 so as to enable further process as further described hereunder.

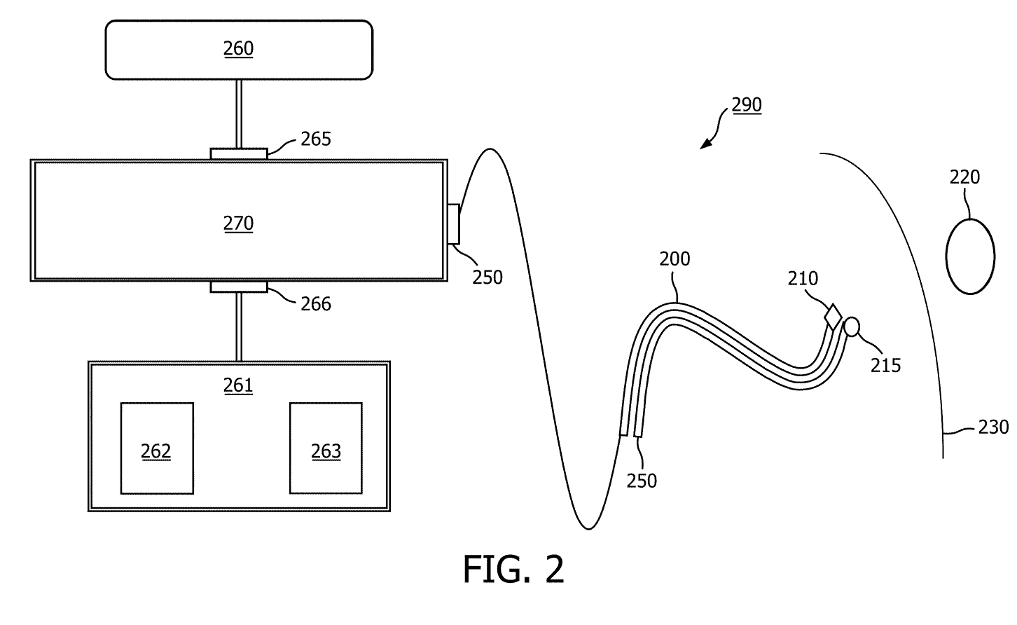

FIG. 2 schematically represents an embodiment of a medical system 290 according to the present invention. This system is arranged to cooperate with a medical device 200 according to the present invention, where said cooperation may be done via one or more wires or alternatively wirelessly (for instance via Bluetooth, Wi-Fi, NFC, ZigBee or any other means capable of transmitting data or information between two or more points that are not connected by an electrical conductor). Any means capable of transmitting information in the form of a signal could enable said cooperation.

The medical system 290 comprises an one or more input(s)/output(s) 250 for enabling the cooperation set forth in the preceding paragraph, such that the information can flow from the medical system 290 to the medical device 200 (for example a control signal), and/or from the medical device 200 to the medical system 290 (for example a detection signal).

The system 229 comprises a receiving unit 265 for receiving data (for instance 2D image data, for instance 3D image data) of the human or animal body wherein the region of interest is identified or identifiable. Those image data may be stored in a memory 261, or alternatively may be received in real time via an imaging modality (not shown) for instance an X-Ray scanner, for instance a MRi scanner, for instance an ultrasound scanner, for instance a CT scanner.

Said medical system 290 comprises further receiving unit 266 configured to receive stored data from the memory 261, said memory 261 configured to store information as to:

a depth (in μm, or nm, or mm, or cm) of a structure within a tissue and/or a cavity of the human or animal body, and

the wavelength, or range of wavelengths that is suitable for illuminating the structure based on the depth,

The information stored therein may have been stored by several means, which are in no way limiting for the present invention.

In an embodiment, the memory 261 is integrated in the system 290. In an alternative embodiment, the memory 261 at a remote location, and accessed by the system 290 according to the present invention. Following identification of the structure of interest 220 and the depth of said structure of interest 220 within a tissue (for instance an organ, for instance a cavity) (from data, such as image data as further detailed hereunder, for instance relative to the surface 230), said structure of interest 220 may be correlated with the structure(s) stored in the memory 261 based on the determined depth such that the wavelength, or range of wavelengths that is suitable for illuminating the structure of interest 220 is determined.

In this exemplary embodiment, the structure of interest 220 is identified in said data (for instance image data) either manually, for instance by a radiologist, additionally or alternatively the structure of interest 220 is identified automatically, for instance following one or more processes by one or more mathematical models. Numerous models may be foreseen by the skilled person so as to automatically process image data such that meaningful information are retrieved therefrom. For instance, statistical shape models, for instance 2D statistical shape models, for instance 3D statistical shape models may be used for automatic detection of shape correspondences. In more details, 3D statistical shape models may enable shape representation, and/or model construction, and/or shape correspondence, and/or local appearance models and/or search algorithms.

For example, the data are then stored in a memory 261 prior to being retrieved by the control unit 270 via the receiving unit 265. The skilled person will foresee several alternative means to determine the depth of the structure of interest 220 relative to the surface of the tissue 230 capable of being illuminated. For instance, the distance between the controllable light source 210 and the wall 230 to be illuminated may be determined. Such distance may have an effect on the wavelength or range of wavelengths that will reach the structure of interest 220. Consequently, the controllable light source 210, or alternatively the control unit 270 may receive such information so that this variable is incorporated in the computation of the wavelength, or range of wavelength to be emitted be said controllable light source 210 thereby enabling illumination of the structure of interest 220 such that said structure of interest 220 becomes visible to the user using a system 229 according to the present invention.

In an exemplary embodiment, the distance between the controllable light source 210 and the structure of interest 220 is determined by establishing the distance between a fixed stopping point (for instance the wall capable of being illuminated 230) and the structure of interest 220. Alternatively, the distance between the controllable light source 210 and the structure of interest 220 is determined by establishing the spatial location of the region of interest (for instance from image data generated from a scan), and using spatial navigation techniques (for instance using a virtual environment such as detailed in Caroline G. L. Cao, Paul Milgram, Direction and location are not sufficient for navigating in nonrigid environments: An empirical study in augmented reality, Presence: Teleoperators and Virtual Environments, Volume 16 Issue 6, December 2007 Pages: 584-602) so that the location of the medical device 200 and/or the controllable light source 210 relative to the structure of interest 220 is computed and therefore determined.

Alternatively, the distance between the controllable light source 210 and the structure of interest 220 is determined by carrying a scan in real-time as the medical device 200 is inserted into a cavity of the body so as to directly measure the location of the medical device 200 and/or the controllable light source 210 relative to the structure of interest 220, such as further detailed in Than, T. Due., Alici, G., Zhou, H. & Li, W. (2012). A review of localization systems for robotic endoscopic capsules. IEEE Transactions on Biomedical Engineering, 59 (9), 2387-2399.

Based on the determined distance between the controllable light source 210 and the structure of interest 220, and additionally or alternatively another environmental parameters (for instance property of the body fluid between the controllable light source 210 and the wall 230), the controllable light source 230 and/or the control unit 270 is configured to determine the wavelength or range of wavelengths to be emitted by the controllable light source 210 so that the structure of interest 220 is illuminated such that said structure of interest 220 becomes visible to the user using a system 229 according to the present invention.

Additionally, or alternatively, the data image may be processed, or have been processed by one or more mathematical models, for instance an image segmentation algorithm, for instance a shape-constrained deformable model, for instance an active shape model such that the region of interest (not shown) is identified into the data (for instance image data), more particularly a contour of the region of interest is enforced, said contour of the region of interest being the schematic visual representation of the contour of the structure of interest 220 on an image, for instance a medical image.

Alternatively, or additionally, the data image may be processed, or have been processed using models-based segmentation configured to detect, or approximate the desired organ using a feature extraction technique, for example a generalized Hough transformation (GHT). A generalized Hough transformation is the modification of the Hough Transform using the principle of template matching. This modification enables the Hough Transform to be used for not only the detection of an object described with an analytic function, but also to detect an arbitrary object described with its model.

In an exemplary embodiment wherein the data image are processed by a generalized Hough transformation, the output of such technique can be used to roughly position a generic organ model that is subsequently adapted to the image. Within the model adaptation process of the generalized Hough transformation, image borders or image contours are detected and the generic organ model is adapted with increasing degrees of freedom (for instance initially rigid, then affine and then deformable). After the model adaptation, the segmented organ surface may be further analyzed. As a non limiting example, profiles perpendicular to the organ surface can be analyzed (e.g. ID model of wall profile and adaptation of parameters of profile) to derive information of the wall thickness of the organ. Tissue classification approaches (for instance thresholding) in combination with region growing may be used within an organ such as the kidney to detect a region of interest, such as a cyst or a tumor. Consequently, the distance of a region of interest relative to the internal wall of the organ, in addition to the thickness of the organ's wall 230 are established such that the information of depth of the structure of interest 220 is assessed and provided to the receiving unit 265 according to the present invention.

In an embodiment, the memory 261 comprises a first look-up table 262 for storing information as to the depth of the region of interest (for instance corresponding to the structure of interest 220) relative to a surface 230 of the tissue capable of being illuminated by the controllable light source 210 and a second look-up table 263 for storing information as to the wavelength, or range of wavelengths that is (are) most suitable for illuminating the structure of interest 220 (for instance corresponding to the region of interest) based on the depth of said structure of interest 220 relative to the surface 230 of the tissue.

The first look-up table 262 may contain records of features for number of given tissues, or organs, or cavity which may be of interest for the endoscope operator in addition to the depth at which different structures (for instance tumor) may be in the tissue, or organ, or cavity. Different alternative ways may be foreseen to organize the information in the first look-up table 262 such as on organs, additionally or alternatively on medical tissue, additionally or alternatively on a patient's medical history, additionally or alternatively via any other classification method that the skilled user will find suitable.

The second look-up table 263 may contain the most appropriate wavelength or range of wavelengths to be emitted by a light source (for instance a controllable light source 210) to illuminate a given structure 220 at a certain depth relative to the surface 230 of a given tissue (for instance an organ, a cavity). This information of said second look-up table 263 is tissue specific, and based on determined anatomical information, associated tissue and other constituent properties.

Additionally or alternatively, the information (for instance the data, for instance the numerical numbers, for instance the numerical range) as to the depth of the region of interest relative to a surface 230 of the tissue capable of being illuminated by the controllable light source 210 and/or the wavelength, or range of wavelengths that is (are) most suitable for illuminating the structure of interest 220 may be stored on a remote server, for instance at a remote location, such that the memory 261 comprises means enabling web-access (such as internet, or intranet, or any other protocol) so as to gain access to such information stored at a remote location and receive such information to be thereafter transferred to the control unit 270.

A correlation between the determined structure of interest 220 and the structure as found in the first and second look-up tables 262, 263 enables an association between said determined region of interest 220 and the wavelength, or range of wavelengths.

The skilled in the art will understand that the correlated information contained in the first and second look-up tables 262, 263 serves so as to assess, alternatively to find, alternatively to calculate the wavelength or range of wavelengths suitable for illuminating a structure, as each tissue has, for example, a different adsorption coefficient. Correlation of the information as to the determined depth of the structure of interest 220 in a given tissue (for instance organ) and the wavelength, or range of wavelengths optimal to illuminate a structure in said given tissue (for instance organ) so as to enable determination of the optimal (or close to optimal) wavelength or range of wavelengths to be send to the controllable light source 210 via the control signal so as to enable illumination of the determined structure of interest 220 by the emitted light from the controllable light source 210. Alternatively, the correlation feature could be done in the medical device 200 such that the controllable light source 210 emits light at a determined wavelength or range of wavelengths.

Amongst different means to have the first and second look-up table 262, 263 contained in the memory 261 able to communicate such correlated information is determined, the present relies for instance in known concept of data transformation services. Such association of information can achieve for instance via a mathematical model, such as an algorithm, for instance via a data transformation services (DTS) such as, for example SQL Server Integration Services (Microsoft Corporation).

As a first exemplary use case of the present invention, the reader will appreciate the following use case.

A patient has a suspected tumor which has been identify following analysis of image data (for instance following an ultrasound) is located 100 mm under the surface of his/her liver. Preferably, said image data has been further processed by an Anatomical Intelligence algorithm such that the region of interest is clearly identified as a tumor, and such that the distance between the wall of the liver and said region of interest is determined (being 100 mm).

In order to gather more information on said tumor, the physician decides to proceed with a minimally invasive surgery, using a medical device according to the present invention, for instance an endoscope.

Namely from the information that the region of interest is located 10 mm from the wall of the liver, the control unit 270 may retrieve relevant information so that an appropriate wavelength (or alternatively a range of wavelength), for instance 630 nm is determined.

An output signal indicative of a wavelength of 630 nm is sent from the control unit 270 to the controllable 310 light source so that said controllable light source emits light at 630 nm on the liver wall between the controllable light source and the structure of interest.

The reflection of the emitted light source is received by one or more sensor (for instance an optical fiber) such that the received signal is adequately processed so that an image is generated and shown on a display.

Additionally, the spectral properties are adapted based on environmental properties such as tissue type, both of the tumor and the tissue between the endoscope and the structure of interest.

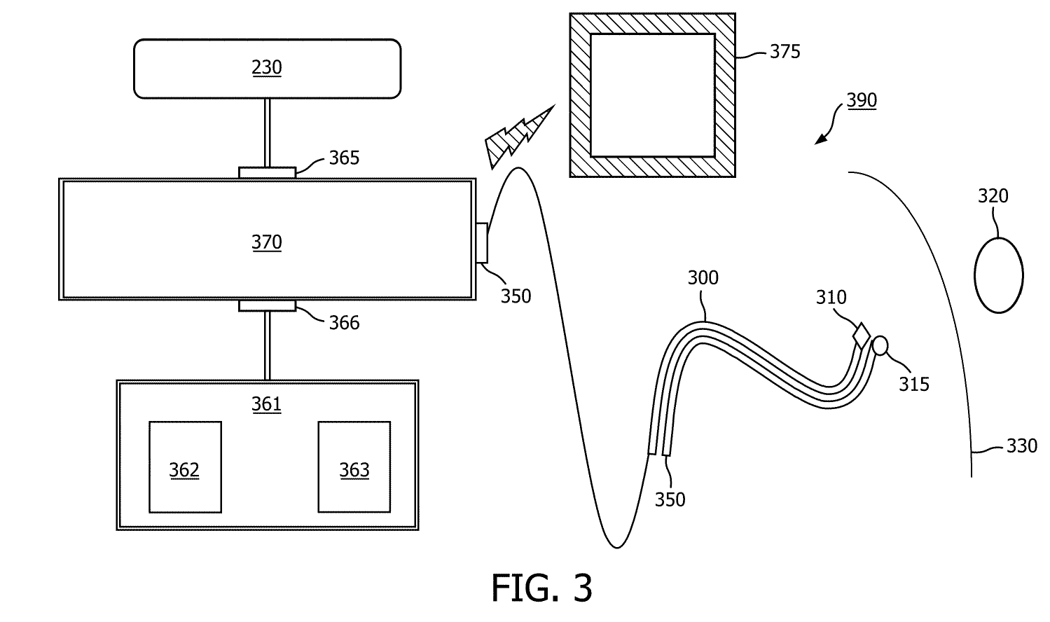

FIG. 3 schematically represents an embodiment of a medical system 390 according to the present invention. This embodiment further comprises a display 375 enabling the user to see for example the structure of interest 310 via the detection signal generated by the sensor 315 arranged to receive reflected light from the structure of interest 320 such that a detection signal is generated and outputted from the medical device 300.

The controllable light source 310 of the medical device 300 according to the present invention is configured, as previously elucidated, to emit light at a wavelength or a range of wavelengths based of a control signal such that a structure of interest 320 is illuminated. Following the physical property of the tissue in which the structure of interest 320 lays, as well as the property of the structure of interest 320, as well as some other properties such as the fluid in between the controllable light source 310 and the surface 330, the emitted light from the controllable light source 310 will be reflected such that the sensor 315 will receive reflected light.

Said reflected light, which will be transmitted via the detection signal is based on the emitted wavelength, or range of wavelengths and can be processed via known imaging processing algorithms and/or software (for instance any of the processing techniques disclosed in Liedlgruber, M., Uhl, A., Endoscopic image processing—an overview, Image and Signal Processing and Analysis, 2009. ISPA 2009. Proceedings of 6^{th} International Symposium on Sep. 16-18, 2009). Once processed, the processed signal may be displayed on a display 375 enabling the user to see, in real time, the structure of interest and any other element of interest (for instance, the contour of said structure of interest following marking with a fluorescent marker).

Additionally, or alternatively the medical system 390 is further comprise a location unit (not shown) for generating a location signal indicative of the location of the controllable light source 310 inside the human or animal body. In this embodiment, the medical system 390 further comprising an alarm generator (not shown) for emitting an alarm when the received location signal indicates that the controllable light 310 source is located so as to illuminate the structure of interest 320. An apparatus comprising a spectrometer for determination of a parameter indicative of tissue type of the associated tissue is further detailed in US 2014/0200459 A1.

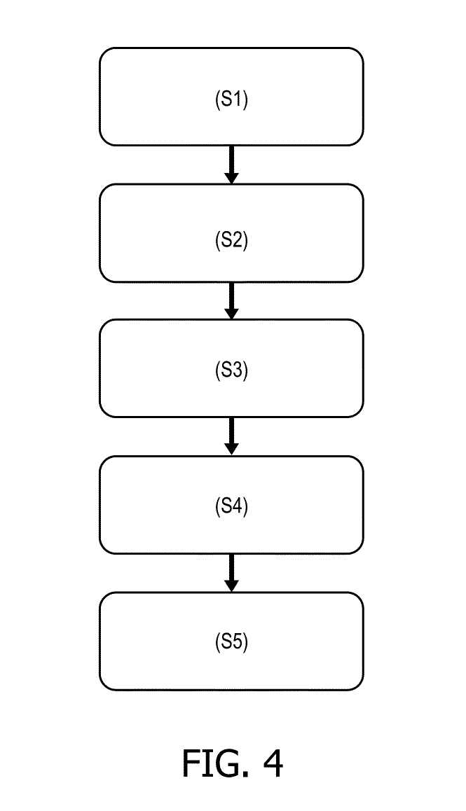

FIG. 4 schematically represents an embodiment of a method according to the present invention. According to this embodiment, the step S1 consists in providing (for instance, inserting) a medical device according to the present invention, preferably such that the medical device is located inside the human or animal body. Said insertion can be invasive, or minimally invasive such that the controllable light source is arranged to illuminate a cavity of the body, additionally or alternatively an organ of the body, additionally or alternatively any other tissue capable of being illuminated by a light source (for instance, a controllable light source).

Step S2 consists in receiving data (for instance, image data) of the human or animal body. Those data can be stored, alternatively or additionally processed, and sent to the system at the required future moment, or alternatively sent in real time so as to achieve the method according to the present invention. In other words, step S2 may comprise receiving stored data, or receiving simultaneously generated (real time) data.

Step S3 consists in determining a suitable wavelength or range of wavelengths based on correlated information contained in a memory as to i) a depth of a structure relative to a surface of a given tissue of the human or animal body, said surface capable of being illuminated by the controllable light source and ii) the wavelength, or range of wavelengths that is suitable for illuminating the structure based on the depth. By associating and/or correlating the information determined in S2, the determination of S3 enables the determination of the wavelength or range of wavelengths based on the information contained in the memory, such information based of the tissue and/or organ and/or density and/or other criterion that may be of relevance.

Step S4 consists in outputting a control signal based on the received image and correlated information, a control signal capable of being received by the controllable light source. Said signal may comprise the wavelength, or the range of wavelengths at which the controllable light source should emit light. Additionally, or alternatively, said control signal provides a processing unit with the relevant information such that the processing unit embedded in a medical device according to the present invention for making the controllable light source emitting light at a wavelength or a range of wavelengths which is suitable to illuminate the structure of interest inside the body. Said structure of interest is hidden by a wall of the tissue, or a wall of the organ that is capable of being illuminated.

Step S5 consists in controlling the controllable light source so to illuminate the structure of interest at a wavelength, or a range of wavelengths suitable to illuminate the structure of interest, said wavelength, or a range of wavelengths being chosen from the selected wavelength, or the selected range of wavelengths, selected from a range of wavelengths, based on the received control signal.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A medical system for illuminating a structure of interest inside a human or animal body, the medical system comprising:
a medical device comprising a controllable light source configured to emit light with a selected wavelength, or a selected range of wavelengths, chosen from a range of wavelengths,
an input for receiving a control signal emitted by a control unit;
wherein the controllable light source is further configured to illuminate the structure of interest at a wavelength, or a range of wavelengths suitable to illuminate the structure of interest, wherein said wavelength, or a range of wavelengths being chosen from the selected wavelength, or the selected range of wavelengths, based on the received control signal,
characterized in that said medical system comprises:
a control unit for generating the control signal for controlling the controllable light source, wherein the control unit comprises:
a receiving unit configured to receive data of the human or animal body, and
a further receiving unit configured to receive stored information from a memory, said memory configured for storing predetermined information as to i) a depth of a structure of interest within a tissue and/or a cavity of the human or animal body, and ii) the wavelength, or range of wavelengths suitable for illuminating the structure of interest based on the depth of said structure of interest within the tissue and/or the cavity,
wherein a given predetermined depth value corresponds to a predetermined plurality of wavelengths, or plurality of ranges of wavelengths for illuminating the structure of interest, wherein a suitable one of the plurality of wavelengths, or of the plurality of ranges of wavelengths is selected based on at least one of a physical property of tissue in which the structure of interest lays, a property of the structure of interest, and a property of a fluid in between the controllable light source and the structure of interest,
whereby the control unit is arranged to generate, based on the received data and the stored information, a control signal capable of being received by the controllable light source such that said controllable light source is arranged to emit light at a wavelength, or a range of wavelengths that is selected as suitable to illuminate said structure of interest based on said control signal.

2. The medical system as claimed in claim 1, wherein the medical device further comprising a detection unit configured to receive reflected emitted light from the illuminated structure of interest, and to generate a detected signal.

3. The medical system as claimed in claim 1, wherein the controllable light source comprises a light-emitting diode (LED), or a distal end of an optical fiber.

4. The medical system as claimed in claim 1, wherein the medical device is an endoscope.

5. The medical system as claimed in claim 1, wherein the received data of the human or animal body comprise region information such that a region of interest is determined, such region of interest corresponding to the structure of interest.

6. The medical system as claimed in claim 5, wherein the received data comprise image data of the region of interest that have been processed by one or more mathematical models prior to be received by the receiving unit, such mathematical model arranged for recognizing and/or segmenting the region of interest in the image data such that the region of interest is automatically identified in the image data.

7. The medical system as claimed in claim 6 wherein the image data have been processed by one or more deformable models such that a contour of the region of interest within an organ is identified in the image data.

8. The medical system as claimed in claim 1, further comprising a correlation unit configured to correlate the received data from the receiving unit and the further receiving unit such that the depth and wavelength, or range of wavelengths are correlated, thereby generating correlated data, wherein the control unit is arranged to generate based on the correlated data the control signal.

9. The medical system as claimed in claim 1, wherein the memory comprises:
a first look-up table comprising information as to the depth of the structure of interest relative to a surface of the tissue, said surface capable of being illuminated by the controllable light source; and
a second look-up table comprising information as to the wavelength, or range of wavelengths that is most suitable for illuminating the structure of interest based on the depth of the structure of interest relative to a surface of the tissue capable of being illuminated by the controllable light source.

10. The medical system as claimed in claim 6, further comprising a display (375) for displaying a visualization of a detected signal generated by the medical device.

11. The medical system as claimed in claim 6, wherein a database is further arranged to host a mathematical model configured to run a correlation analysis on the information contained in said memory such that the control unit emits a control signal based on the correlated analysis.

12. A method for calculating an illumination for a structure of interest inside a human or animal body, the method comprising the steps of:
providing a medical device according to claim 4;
the method being characterized in the steps of:
receiving data of the human or animal body;
determining a suitable wavelength or range of wavelengths based on correlated information contained in a memory as to i) a depth of a structure of interest within a tissue and/or a cavity of the human or animal body, and ii) the wavelength, or range of wavelengths suitable for illuminating the structure of interest based on the depth of said structure of interest within the tissue and/or the cavity, wherein a given predetermined depth value corresponds to a predetermined plurality of wavelengths, or plurality of ranges of wavelengths for illuminating the structure of interest, and wherein a suitable one of the plurality of wavelengths, or of the plurality of ranges of wavelengths is selected based on at least one of a physical property of tissue in which the structure of interest lays, a property of the structure of interest, and a property of a fluid in between the controllable light source and the structure of interest;
outputting a control signal based on the received image and correlated information, a control signal capable of being received by the controllable light source to control the controllable light source based on the received control signal so as to emit light at a wavelength, or a range of wavelengths suitable to illuminate the structure of interest, said wavelength, or a range of wavelengths being chosen from the selected wavelength, or the selected range of wavelengths, selected from a range of wavelengths.

13. A computer program comprising program code means for causing, when executed, a medical system for calculating an illumination for a structure of interest inside a human or animal body as claimed in claim 12.
